(19) United States
(12) Patent Application Publication
(10) Pub. No.: US 2024/0280563 A1
(43) Pub. Date: Aug. 22, 2024

(54) SYSTEMS AND METHODS FOR ION CHANNEL KINETICS ANALYSIS IN CLUSTERS OF ION CHANNELS

(71) Applicant: Ohio State Innovation Foundation, Columbus, OH (US)

(72) Inventors: Mikhail Tarasov, Columbus, OH (US); Przemyslaw Radwanski, Columbus, OH (US)

(21) Appl. No.: 18/442,534

(22) Filed: Feb. 15, 2024

Related U.S. Application Data
(60) Provisional application No. 63/445,863, filed on Feb. 15, 2023.

(51) Int. Cl. G01N 33/50; G16C 20/10

(52) U.S. Cl. CPC G01N 33/502; G16C 20/10

(57) ABSTRACT

Systems and methods for ion channel kinetics analysis in clusters of ion channels are described herein. In some implementations, the techniques described herein relate to a computer-implemented method including: receiving a multichannel activity signal associated with a plurality of ion channels of a cell; processing the multichannel activity signal to remove a capacitive transient; and analyzing the processed multichannel activity signal to assess cooperative ion channel gating behavior for the plurality of ion channels.

### CROSS-REFERENCE TO RELATED APPLICATIONS

This application claims the benefit of U.S. provisional patent application No. 63/445,863, filed on Feb. 15, 2023, and titled “SYSTEMS AND METHODS FOR IDENTIFICATION OF ION CHANNELS,” the disclosure of which is expressly incorporated herein by reference in its entirety.

### STATEMENT REGARDING FEDERALLY FUNDED RESEARCH

This invention was made with government support under Grant no. NS121234 and HL155378 awarded by the National Institutes of Health. The government has certain rights in the invention.

### BACKGROUND

Na+ channels (Naᵥs) clustering increases conduction velocity. The cytoskeleton and the associated anchoring proteins which control Naᵥs clustering are capable of directly modulating channel kinetics. It is desirable to provide a system and method for analyzing whether Naᵥs that operate in clusters exhibit different channel kinetics relative to those operating outside of clusters.

### SUMMARY

Systems and methods for ion channel kinetics analysis in clusters of ion channels are described herein. In some implementations, the techniques described herein relate to a computer-implemented method including: receiving a multichannel activity signal associated with a plurality of ion channels of a cell; processing the multichannel activity signal to remove a capacitive transient; and analyzing the processed multichannel activity signal to assess cooperative ion channel gating behavior for the plurality of ion channels.

In some implementations, the step of processing the multichannel activity signal to remove the capacitive transient includes applying a capacitive current subtraction algorithm.

In some implementations, the step of analyzing the processed multichannel activity signal includes detecting a time sequence of switches between a plurality of conductance levels. Optionally, the time sequence of switches between the plurality of conductance levels is determined using a Bayesian model.

In some implementations, the step of analyzing the processed multichannel activity signal further includes performing a statistical analysis to assess cooperative ion channel gating behavior for the plurality of ion channels. The statistical analysis is based, at least in part, on the time sequence of switches between the plurality of conductance levels.

In some implementations, the method further includes measuring one or more single channel activity amplitudes present in the multichannel activity signal. Optionally, the one or more single channel activity amplitudes are measured by fitting one or more Gaussian mixture models to the multichannel activity signal.

In some implementations, the method further includes inferring a plurality of ion channel gate state transition probabilities of discrete time Markov models for the multichannel activity signal. Optionally, the plurality of ion channel gate state transition probabilities of discrete time Markov models for the multichannel activity signal are inferred using a Bayesian model.

In some implementations, the multichannel activity signal is measured using a cell-attached patch-clamp system.

In some implementations, the techniques described herein relate to a method including: applying a drug, compound, or agent to a cell; recording a multichannel activity signal associated with a plurality of ion channels of the cell, where the cell has been exposed to the drug, compound, or agent; analyzing ion channel kinetics as described herein; and screening the drug, compound, or agent based, at least in part, on the assessed cooperative ion channel gating behavior for the plurality of ion channels.

In some implementations, the techniques described herein relate to a method including: applying a drug, compound, or agent to a cell; recording a multichannel activity signal associated with a plurality of ion channels of the cell, where the cell has been exposed to the drug, compound, or agent; and screening the drug, compound, or agent based, at least in part, on a cooperative ion channel gating behavior for the plurality of ion channels.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a flow chart illustrating example operations for analyzing ion channel kinetics according to an implementation described herein.

FIG. 2 is an example computing device.

FIG. 3 illustrates representative examples of patch clamp recordings idealization with the Bayesian multiple switch point detection algorithm according to an example described herein.

FIG. 4 illustrates detection of cooperative gating in multichannel hNaᵥ1.6 recordings according to an example described herein.

FIGS. 5A-5B illustrate Bayesian inference of state transition probabilities of discrete time Markov models for single hNaᵥ1.6 channel recordings according to an example described herein.

FIG. 6 illustrates Bayesian inference of state transition probabilities of discrete time Markov models for three hNaᵥ1.6 channel recordings according to an example described herein.

FIGS. 7A-7D illustrate the clustering dependence of NaV1.5 pharmacological blockade according to an example described herein. FIGS. 7A and 7B are representative sets of overlapped current sweeps from membrane patches exhibiting activity of only one (FIG. 7A, left, single channel patch) and more than one (FIG. 7B, left, multichannel patch) channels in control (upper, black) and after application lidocaine (bottom, grey, 50 Um, 5 minute incubation). Voltage protocol is shown in FIG. 7A, left. A number of active channels is determined with fitting gaussian mixture distribution probability density functions (pdf, solid curves in rights panels in FIG. 7A and FIG. 7B) to histograms of all current amplitudes (areas under curves) in sets of 1000 current sweeps (histogram bin width is 0.01 Pa, insets show enlarged regions near 0 of pdf^{1/2}). FIG. 7A and FIG. 7B panels demonstrate paired experiments. FIG. 7C illustrates ensemble average currents obtained from idealized current sweeps shown in FIG. 7A and FIG. 7B in control (black) and after lidocaine (grey) in single channels (left) and multichannel (right) patch. FIG. 7D illustrates a summary of peak current blockade with lidocaine in single channel and multichannel patches expressed as a ration on ensemble average peak current after lidocaine (Ilido) to that before lidocaine (Ictr). 3 and 4 cells for single channels and multichannel patches, respectively. * p<0.05 with unpaired t-test.

FIGS. 8A-8B illustrate a statistical analysis of gating independence in multichannel Naᵥ1.5 patch clamp records. FIG. 8A shows Ensemble average currents (I, mean±standard error of the mean, (S.E.M.), upper), Kullback-Leibler divergence (D_{KL}, middle) and relations between peak current amplitudes and D_{KL} measured in the same membrane patches (bottom) in stochastic simulations of independent Naᵥ1.5 activity (model: 49 simulation of activity of 2 to 150 independent channels generated with the single Naᵥ1.5 channel kinetic model), patch clamp cell-attached records in CHO cells stably expressing human Naᵥ1.5 channels only (Naᵥ1.5), transiently co-transfected with human β1 (Scn1B) NaV auxiliary subunit (Naᵥ1.5+β1), or treated with paclitaxel (TXL, Naᵥ1.5+TXL, 100 μM, 3 hours pre-incubation). Box-end-whiskers plots of peak I and D_{KL} at peak I in all conditions are in insets, *q<0.05 with Kruskal-Wallis test with post-hoc pairwise comparison with Benjamini-Hochberg false discovery rate procedure. Numbers of cells are shown in the plots. D_{KL} is a measure of statistical distance between distribution of observed numbers of open channels relative to best-fit binomial distribution (model of numbers of open channels in identical and independently gating ion channels) parametrized with the standard non-stationary noise analysis. Relations between peak I and D_{KL} at peak I are smoothed with spline curve (dashed line) which is compared to linear regression (solid line) ±95% confidence interval (C.I.). The comparison demonstrates that linear regression is an appropriate model of this relation. FIG. 8B is a summary of relations between peak I and D_{KL} at peak I. Upper: box plot of linear regression slopes (best-fit+standard error (S.E)), Middle and bottom: the relations in all experimentally obtained measurements (Naᵥ1.5, Naᵥ1.5+Beta1, Naᵥ1.5+TXL pooled) in comparison to measurements in stochastic simulations (dashed lines—smoothing spline, solid line—linear regression±95 C.I., middle) and corresponding linear regression slopes (bottom), *p<0.05 with F-test.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. While implementations will be described for analyzing ion channel kinetics of sodium channels, it will become evident to those skilled in the art that the implementations are not limited thereto, but are applicable for analyzing ion channel kinetics of other ion channels including other voltage-gated ion channels, ligand-gated ion channels such as calcium-activated potassium channels, acetylcholine-gated ion channels such as acetylcholine-gated potassium channels, hyperpolarization-activated, nucleotide-gated channels (HCN), etc.

As used herein, the terms “about” or “approximately” when referring to a measurable value such as an amount, a percentage, and the like, is meant to encompass variations of +20%, +10%, +5%, or +1% from the measurable value.

The term “artificial intelligence” is defined herein to include any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes' classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders. The term “deep learning” is defined herein to be a subset of machine learning that that enables a machine to automatically discover representations needed for feature detection, prediction, classification, etc. using layers of processing. Deep learning techniques include, but are not limited to, artificial neural network or multilayer perceptron (MLP).

As used herein, “ion channel the term kinetics” refers the processes that govern the opening and closing (activation and inactivation) of ion channels in biological systems. Ion channels are integral membrane proteins that allow the selective passage of ions across cell membranes, influencing the electrical properties of cells. The kinetics of ion channels can be described in terms of the rates at which these channels undergo transitions between different states. The primary states of interest are typically open, closed, and inactivated. Understanding ion channel kinetics is crucial in fields such as physiology, pharmacology, and neuroscience because these kinetics influence the electrical activity of cells. Manipulating ion channel kinetics can have significant physiological and pharmacological implications, as it can impact processes like action potential generation, neurotransmission, and muscle contraction.

As used herein, the term “ion channel clustering” is the phenomenon where ion channels, particularly in the context of cell membranes, are organized or grouped together in specific regions or domains. This clustering has important implications for cell function, signal transduction, and cellular communication. In other words, ion channel clustering is non-independent gating behavior, where one ion channel effects the gating behavior of one or more other ion channels. Ion channel clustering is sometimes described herein as cooperative ion channel gating behavior.

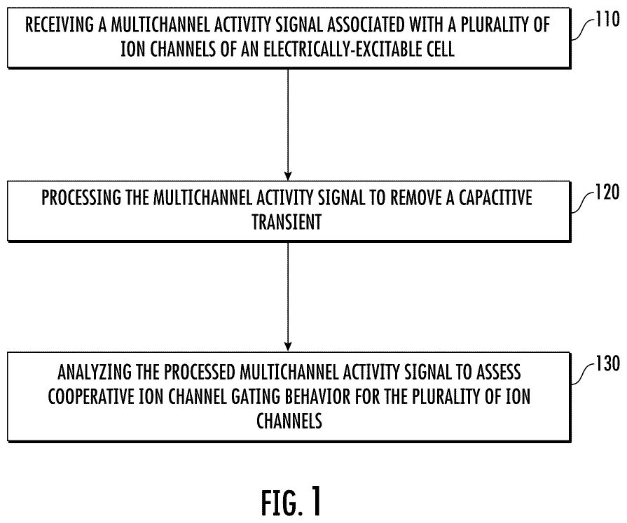

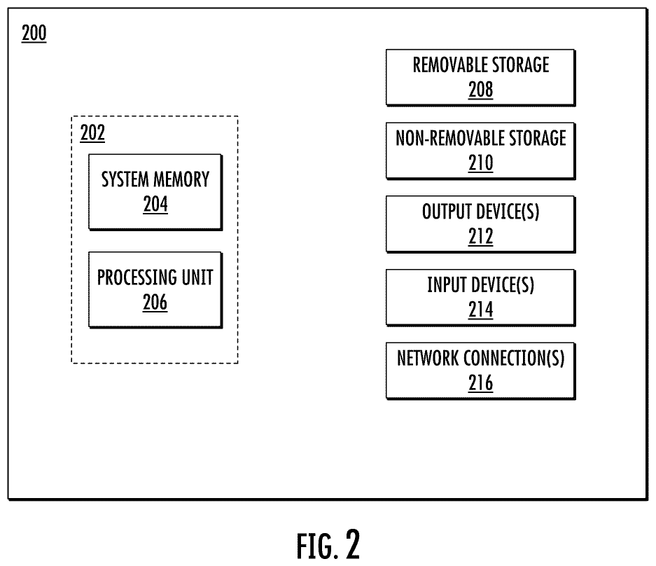

Referring now to FIG. 1, a flow chart illustrating example operations for analyzing ion channel kinetics is shown. This disclosure contemplates that the operations shown in FIG. 1 can be performed using a computing device (e.g., computing device 200 of FIG. 2). The method of FIG. 1 provides an approach for analyzing ion channel kinetics that is not possible using conventional technology. The method of FIG. 1 can be used to assess cooperative ion channel gating behavior. This disclosure contemplates that such information can be used for drug discovery, e.g. identifying clustering-dependent mechanisms of action. This is not possible using conventional drug discovery techniques. For example, ion channel (e.g. sodium channel (Nav)) cooperativity cannot be detected using conventional techniques such as whole cell patch clamp measurements. The method of FIG. 1 address this challenge through a statistical approach. In particular, multichannel patch clamp records and analysis as described herein allow for quantification of Markov model parameters of channels operating in clusters. Comparison of these parameters for channels recorded in control condition with those in the presence of a candidate drug (e.g. a potential antagonist of cooperativity) facilitates making a concussion about effect of the candidate drug on cooperativity of channels behavior.

At step 110, the method includes receiving a multichannel activity signal associated with a plurality of ion channels of a cell. In some implementations, the cell is an electrically-excitable cell. As described herein, a multichannel activity signal captures ion channel gating (e.g., open state to closed state transition and/or vice versa) for a plurality of ion channels. Thus, the multichannel activity signal captures activity for a cluster (i.e., more than 1) ion channels. In the Examples described herein, the ion channels are voltage-gated ion channels. Voltage-gated ion channels include, but are not limited to, a sodium channel (Nav), a potassium channel (Kv), a calcium channel (Cav), or a chloride channel (CIC). Alternatively or additionally, the ion channels may be one of a plurality of sodium ion channel forms, e.g., Nav1.5, Nav1.6, etc. Alternatively or additionally, the ion channels may be one of a plurality of potassium ion channel forms. Alternatively or additionally, the ion channels may be one of a plurality of calcium ion channels. Alternatively or additionally, the ion channels may be one of a plurality of chloride ion channel forms. It should be understood that sodium channels, potassium channels, calcium channels, and chloride channels are provided only as example voltage-gated ion channels. This disclosure contemplates using the method described with respect to FIG. 1 with other voltage-gated ion channels. Alternatively, this disclosure contemplates using the method described with respect to FIG. 1 with other ion channels, which include, but are not limited to, ligand-gated ion channels, acetylcholine-gated ion channels, and HCNs. Additionally, as described in the Examples below, the multichannel activity signal can be measured using a cell-attached patch-clamp system or other known technology. The cells in the Examples below are Chinese Hamster Ovary (CHO) cells. It should be understood that CHO cells are only provided as a non-limiting example of electrically-excitable cells.

At step 120, the method includes processing the multichannel activity signal to remove a capacitive transient. For example, a capacitive current subtraction algorithm can be applied to remove the capacitive transient. Such processing is described in detail in the Examples, Part 3 below.

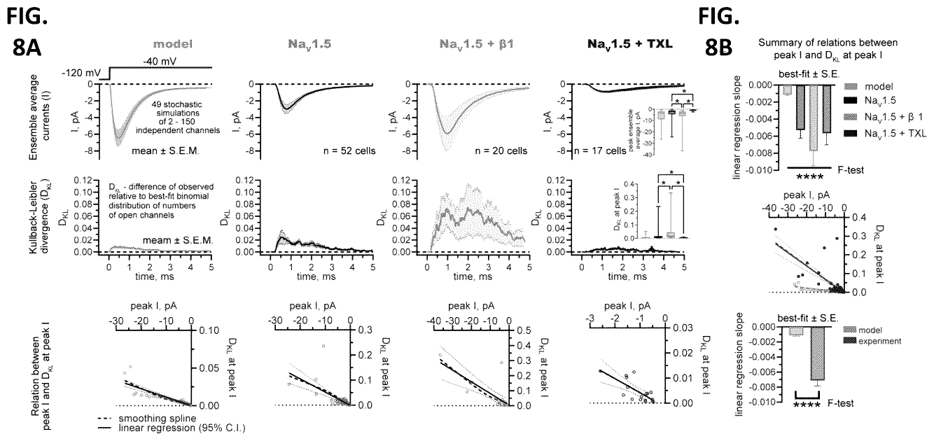

At step 130, the method includes analyzing the processed multichannel activity signal to assess cooperative ion channel gating behavior for the plurality of ion channels. As described herein, cooperative ion channel gating is non-independent gating (e.g., open state to closed state transition and/or vice versa). In other words, the gating behavior of one ion channel effects the gating behavior of one or more other ion channels. The step of analyzing the processed multichannel activity signal can include detecting a time sequence of switches (sometimes referred to herein as “transitions”) between a plurality of conductance levels. Optionally, the time sequence of switches between the plurality of conductance levels is determined using a Bayesian model. Such analysis is described in detail in the Examples, Part 5 below (e.g., a Bayesian multiple switch point detection algorithm). Additionally, the step of analyzing the processed multichannel activity signal can further include performing a statistical analysis to assess cooperative ion channel gating behavior for the plurality of ion channels. The statistical analysis is based, at least in part, on the time sequence of switches between the plurality of conductance levels. Such analysis is described in detail in the Examples, Part 6 below. Statistical analysis of ion channel gating suggests that there is gating cooperativity in experimentally recorded NaV1.5 channels as demonstrated by comparison to in silico simulated independent NaV1.5 gating. Further, cooperativity has a tendency to be more prominent in larger NaV1.5 clusters as shown with effects of Beta1 and TXL in FIGS. 8A and 8B as well as subsequent leaner regression analysis. Together, this indicate that biophysical interaction between NaV1.5 channels exists and can underline reduced sensitivity to lidocaine observed in multichannel membrane patches.

In some implementations, the method optionally further includes measuring one or more single channel activity amplitudes present in the multichannel activity signal. Optionally, the one or more single channel activity amplitudes are measured by fitting one or more Gaussian mixture models to the multichannel activity signal. Such measurement is described in detail in the Examples, Part 4 below.

In some implementations, the method further includes inferring a plurality of ion channel gate state transition probabilities of discrete time Markov models for the multichannel activity signal. Optionally, the plurality of ion channel gate state transition probabilities of discrete time Markov models for the multichannel activity signal are inferred using a Bayesian model. Such inference is described in detail in the Examples, Part 7 below.

This disclosure contemplates that the techniques described with regard to FIG. 1 can be used to screen compounds. For example, a method can include applying a drug, compound, or agent to a cell; recording a multichannel activity signal associated with a plurality of ion channels of the cell, where the cell has been exposed to the drug, compound, or agent; analyzing ion channel kinetics as described with regard to FIG. 1; and screening the drug, compound, or agent based, at least in part, on the assessed cooperative ion channel gating behavior for the plurality of ion channels. For example, using the techniques described herein, it is possible to identify a cluster-dependent mechanism of action.

In some implementations, the techniques described herein relate to a method including: applying a drug, compound, or agent to a cell; recording a multichannel activity signal associated with a plurality of ion channels of the cell, where the cell has been exposed to the drug, compound, or agent; and screening the drug, compound, or agent based, at least in part, on a cooperative ion channel gating behavior for the plurality of ion channels. For example, using the techniques described herein, it is possible to identify a cluster-dependent mechanism of action.

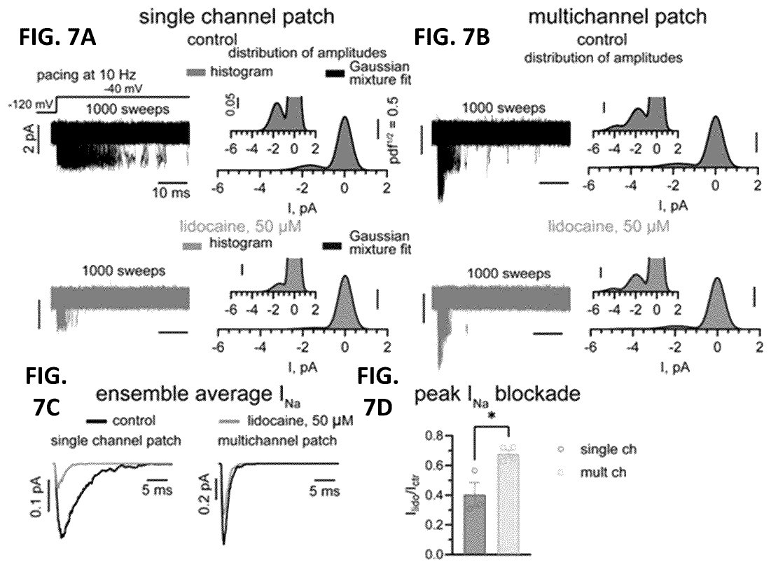

Cooperative behavior of ion channels can modulate potency of clinically relevant drugs. with reference to FIGS. 7A-7D, experiments were performed to assay potency of the prototypical, clinically relevant NaVs blocker lidocaine on single channel membrane patches in comparison to multichannel membrane patches. It was found that potency of lidocaine was ˜40% lower for channel operating in clusters relative to channels operating outside of clusters. This suggests that cooperativity between NaVs in clusters can reduce potency of NaVs blockers. This finding is in line with Zheng Y, Deschênes I. Protein 14-3-3 Influences the Response of the Cardiac Sodium Channel Naᵥ1.5 to Antiarrhythmic Drugs. J Pharmacol Exp Ther. 2023 March; 384(3):417-428. doi: 10.1124/jpet.122.001407. Epub 2022 Dec. 2. PMID: 36460339; PMCID: PMC9976794, which considered the effect of another NaV blocker quinidine and its dependence on difopein which is assumed to be an antagonist of NaVs cooperativity.

Importantly, this and previous studies (e.g. Clatot J, Hoshi M, Wan X, Liu H, Jain A, Shinlapawittayatorn K, Marionneau C, Ficker E, Ha T, Deschênes I. Voltage-gated sodium channels assemble and gate as dimers. Nat Commun. 2017 Dec. 12; 8(1):2077. doi: 10.1038/s41467-017-02262-0. PMID: 29233994; PMCID: PMC5727259) demonstrate that disruption of cooperativity with difopein does not change significantly whole cell sodium current density. Taken together, this suggests that pharmacological agents antagonizing NaVs cooperativity could be harnessed to increase efficiency of therapeutic NaVs blockers (for example, lidocaine and quinidine) without significant effects on basic electrophysiological properties of excitable membranes.

However, difopein remains the only known candidate substance for NaVs cooperativity disruption. Additionally, therapeutic usage of difopein is problematic due to its chemical nature (a membrane impermeable peptide) and the indirect effect on NaVs (it blocks the ubiquitous protein-protein interaction mediator 14-3-3 protein, thus, can have many side effects). Therefore, there is a need for search of other candidate drugs capable of NaV cooperativity disruption. But this search cannot be performed using the standard tools based on measurements of whole cell currents since, as mentioned above, disruption of cooperativity is not expected to change whole cell sodium currents. This calls for methods and systems as described herein, which allow for detection of effects of drugs on cooperative behavior in sodium channels clusters.

In summary, this disclosure provides a tool for detection of presence of cooperativity in NaVs behavior. This tool can be used for screening drugs reducing NaVs cooperativity (e.g. searching for a better alternative of difopein). These drugs in their turn can be used together with therapeutic NaVs blockers (for example, lidocaine, quinidine) to enhance the efficiency of the latter.

It should be appreciated that the logical operations described herein with respect to the various figures may be implemented (1) as a sequence of computer implemented acts or program modules (i.e., software) running on a computing device (e.g., the computing device described in FIG. 2), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 2, an example computing device 200 upon which the methods described herein may be implemented is illustrated. It should be understood that the example computing device 200 is only one example of a suitable computing environment upon which the methods described herein may be implemented. Optionally, the computing device 200 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 200 typically includes at least one processing unit 206 and system memory 204. Depending on the exact configuration and type of computing device, system memory 204 may be volatile (such as random access memory (RAM)), non-volatile (such as read-only memory (ROM), flash memory, etc.), or some combination of the two. This most basic configuration is illustrated in FIG. 2 by box 202. The processing unit 206 may be a standard programmable processor that performs arithmetic and logic operations necessary for operation of the computing device 200. The computing device 200 may also include a bus or other communication mechanism for communicating information among various components of the computing device 200.

Computing device 200 may have additional features/functionality. For example, computing device 200 may include additional storage such as removable storage 208 and non-removable storage 210 including, but not limited to, magnetic or optical disks or tapes. Computing device 200 may also contain network connection(s) 216 that allow the device to communicate with other devices. Computing device 200 may also have input device(s) 214 such as a keyboard, mouse, touch screen, etc. Output device(s) 212 such as a display, speakers, printer, etc. may also be included. The additional devices may be connected to the bus in order to facilitate communication of data among the components of the computing device 200. All these devices are well known in the art and need not be discussed at length here.

The processing unit 206 may be configured to execute program code encoded in tangible, computer-readable media. Tangible, computer-readable media refers to any media that is capable of providing data that causes the computing device 200 (i.e., a machine) to operate in a particular fashion. Various computer-readable media may be utilized to provide instructions to the processing unit 206 for execution. Example tangible, computer-readable media may include, but is not limited to, volatile media, non-volatile media, removable media and non-removable media implemented in any method or technology for storage of information such as computer readable instructions, data structures, program modules or other data. System memory 204, removable storage 208, and non-removable storage 210 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 206 may execute program code stored in the system memory 204. For example, the bus may carry data to the system memory 204, from which the processing unit 206 receives and executes instructions. The data received by the system memory 204 may optionally be stored on the removable storage 208 or the non-removable storage 210 before or after execution by the processing unit 206.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

The method includes of the following parts:
Part 1: Patch clamp registrations.
Part 2: Patch clamp recordings preprocessing in Clampfit (Molecular Devices).
Part 3: Capacitive current subtraction with the convex optimization algorithm.
Part 4: Measuring of single channel current amplitudes.
Part 5: Patch clamp recordings idealization with the Bayesian multiple switch point detection algorithm.
Part 6: Detection of cooperative gating in multichannel recordings.
Part 7: Bayesian inference of state transition probabilities of discrete time Markov models for single- and multi-channel recordings.

### Part 1: Patch Clamp Registrations

Cell types: a cell line with heterologous expression of ion channels of interest, cardiac myocytes.

Patch clamp mode: Voltage clamp.

Patch clamp configuration: Cell attached.

Pipette solution (mM): 280 NaCl, 4 CsCl, 1 CaCl₂), 1 MgCl₂, 10 HEPES, 0.05 CdCl2, pH 7.4 with CsOH.

Bath solution (mM): 140 KCl, 2 CaCl₂), 1 MgCl₂, 10 HEPES, pH 7.4 with KOH (for a cell line),

20 KOH, 120 KCl, 2 CaCl₂), 10 EGTA, 1 MgCl₂, 10 HEPES, pH 7.4 with KOH (for cardiac myocytes).

Pipettes fabrication: (1) pull pipettes from thick borosilicate glass capillary, (2) coat pipettes with Sylgard 184 silicone elastomer, (3) fire polish pipette tips.

Pipette resistance: 2-5 MOhm for low level expression ion channels in a cell line cells, 5-15 MOhm for high level expression ion channels in a cell line, 4-6 MOhm for cardiac myocytes.

Low pass filter: 8 pole Bessel filter, cut off frequency 4 kHz.

Acquisition rate: 100 kHz.

Voltage protocol: Step voltage protocol. Holding potential 80 mV, pre-pulse to 120 mV for 200 ms, test potential to the potential of maximal channel activity (e.g. 10 mV for Naᵥ1.6, 40 mV for NaV1.5, 40 mV for cardiac myocytes) for 1 s, time between sweeps 3 s at holding potential, minimal number sweeps 100.

### Part 2: Patch Clamp Recordings Preprocessing in Clampfit (Molecular Devices)

Remove unstable sweeps.

Adjust baseline for all sweeps by subtracting their average current amplitudes during period of low ion channel activity lasting not less than 200 ms.

Reduce data sampling frequency by factor 5 (down sample from 100 kHz to 20 kHz).

Save resulting file.

Part 3: Capacitive Current Subtraction with the Convex Optimization Algorithm

The capacitive current subtraction algorithm is iteratively applied to each sweep of a preprocessed patch clamp recording. The algorithm is applied for the 20 ms period of a recording during the test potential application starting from 0.5 ms following test potential onset. Current trace during the specified period is a function:

$$I = I ( t ) ,$$

where I—current (pA), t—discrete time (ms): (t₀, . . . , tᵢ, . . . , t_{T-1}), t₀=0, T—total number of time points.

The function is assumed to be equal:

$$I ( t ) = s ( t ) + {\sum }_{j = 1}^{K} {a}_{j} {e}^{\frac{- t}{{τ}_{j}}} ,$$

where s(t)—function representing ion channel currents at each time point tᵢ, then s—vector representing values of ion channel current amplitudes at each time point tᵢ: s=(s₀, s₁, . . . , sᵢ, . . . , s_{T-1}), aⱼ—amplitude parameter of the j-th exponential decay component with time constant Tj, then vector a=(a₁, a₂, . . . , aⱼ, . . . , a_{K}), K—total number of exponential decay components.

Next, Adam optimization algorithm (learning rate of 0.1, the number of optimization steps 250) is applied to infer optimal s and a with following constrained minimization:

$${min}_{a , s} ( \frac{1}{2} {\vert I - {\sum }_{j = 1}^{K} {a}_{j} {e}^{\frac{- t}{{τ}_{j}}} \vert}_{2}^{2} + λ {\vert s \vert}_{1} ) s . t . {s}_{i} \leq 0 , {a}_{j} \geq 0 ,$$

where λ—the L1 regularization penalty term equal 0.1. The optimal time constants τⱼ are following (ms): 0.5, 1, 2, 3, 4, 5, 7, 10, 50.

After optimization and finding the optimal vector of exponential decay amplitude components â=(â₁, â₂, . . . , âⱼ, . . . , â_{K}), the resulting current trace I_{final} is calculated as following:

$${I}_{final} = I - {\sum }_{j = 1}^{K} {\hat{a}}_{j} {e}^{\frac{- t}{{τ}_{j}}} .$$

### Part 4: Measuring of Single Channel Current Amplitudes

Single channel current amplitudes are measured with fitting Gaussian mixture models to current sweeps after capacitive current subtraction by means of the expectation-maximization (EM) algorithm. Current amplitudes (I) within one current sweep are assumed to be generated from univariate Gaussian mixture model with the following probability density function:

$$f ( I ) = {\sum }_{i = 1}^{K} {ϕ}_{i} N ( {μ}_{i} , {σ}_{i}^{2} ) ,$$

where ϕᵢ—weights of gaussian components i∈ (1, . . . , K), N(μᵢ, σᵢ²)—probability density function of i-th gaussian component with mean μᵢ and variance σᵢ². μᵢ corresponds to a current amplitude of a conductance level observed during a sweep, and σᵢ² determines the current noise at this conductance level. Then K equals to the total number of conductance levels present within an analyzed current sweep.

To find optimal K for each current sweep sequential EM fitting of gaussian mixture models with increasing K (from 1 to 10) is applied to each current sweep. The time period for the gaussian mixture fit is first 50 ms interval of a current sweep. Then an optimal model is chosen as the model having the lowest value of Bayesian information criterion (BIC) among all fitted models.

Next, μᵢ from each current sweep with optimal K>1 are sorted and the absolute values of differences between neighbor values |μᵢ₊₁−μᵢ| are defined as single channel current amplitudes.

Next, single channel current amplitudes are estimated from analyzed sweeps within a recording from one membrane patch are used to calculate the mean single channel current amplitude (Iᵤₙᵢₜ):

$${I}_{u n i t} = \frac{1}{W} {\sum }_{w = 1}^{W} [ \frac{1}{{K}_{w}} {\sum }_{i = 1}^{{K}_{w}} \vert {μ}_{i + 1} - {μ}_{i} \vert ] ,$$

where j=1, . . . , W—current sweeps with K>1, and K_{w}—total number of conductance levels in w-th sweep.
   Part 5: Patch Clamp Recordings Idealization with the Bayesian Multiple Switch Point Detection Algorithm

A cell attached patch clamp current sweep is considered as a time series of immediate transitions (switches) between different conductance levels S=(0, 1, . . . , sᵢ, . . . , N−1), N—total number of conductance levels. The transitions between conductance levels are caused by gating of ion channels operating in the membrane patch. Particularly, 0-th conductance level corresponds to 0 open channels, 1-th conductance level—1 open channel, and so on. The transitions between conductance levels are buried in the current noise. The goal of idealization is to recover the time sequence of conductance levels from noise. This is achieved with Bayesian inference of optimal sequence of conductance levels during a recording: x=(Sᵢ_{₀}, . . . , Sᵢ_{₁}, . . . , Sᵢ_{_{T-1}}), t∈(0, 1, . . . , t, . . . , T−1), sᵢ_{ₜ}∈S, t—a time point, sᵢ_{ₜ}—conductance level at a time point t, T—the total number of time points within the analyzed recording. The following Bayesian model is used to perform this inference:

$$\begin{matrix}N > 1 , i , j ∈ ( 0 , 1 … , N - 1 ) , \\ {μ}_{i n i {t}_{i}} = {s}_{i} {I}_{u nit} , \\ {μ}_{i} ∼ 𝒩 ( {μ}_{i n i {t}_{i}} , {σ}_{μ}^{2} ) , \\ {σ}_{n o {ise}_{i}}^{2} ∼ 𝒩 ( {μ}_{{σ}_{n o ise}^{2}} , 1 ) , \\ {p}_{i j , i \neq j , i = 0} = {p}_{base} , \\ {p}_{i j , i \neq j , i > 0} = {p}_{b a s e} \sqrt{c oef \cdot {s}_{i}} , \\ {p}_{i j , i = j} = 1 - {\sum }_{j = 0}^{N - 1} {p}_{i j , i \neq j} , \\ {p}_{i} = ( {p}_{i 0} , … , {p}_{i j} , … , {p}_{i ( N - 1 )} ) , \\ I = ( {I}_{0} , … , {I}_{t} , . . {I}_{T - 1} ) , \\ I ∼ HMM ( 𝒰 ( 0 , N - 1 ) , Cat ( {p}_{i} ) , 𝒩 ( {μ}_{i} , {σ}_{n o {ise}_{i}}^{2} ) ) ,\end{matrix}$$

where i and j—indices of conductance levels S=(0, 1, . . . , sᵢ, . . . , N−1)=(0, 1, . . . , sⱼ, . . . , N−1), N—the total number of conductance levels, μᵢₙᵢₜ_{ᵢ}—a prior estimation of a current amplitude at i-th conductance level, Iᵤₙᵢₜ—the mean single channel current amplitude measured as described in section 4, μᵢ—a current amplitude at the i-th conductance level, [symbol] (μᵢₙᵢₜ_{ᵢ}, σ_{μ}²)—normal distribution with the mean μᵢₙᵢₜ_{ᵢ} and the variance σ_{μ}². A value of σ_{μ}² depends on the noise in a particular patch clamp setup and varies between 0.05-0.5. σₙₒᵢₛₑ_{ᵢ}²—the variance of the current noise at i-th conductance level assuming the current noise is gaussian at all conductance levels but amplitudes of the noise could be different for different conductance levels, [symbol] (μ_{σ}_{ₙₒᵢₛₑ}_{²},1)—normal distribution with the mean μ_{σ}_{ₙₒᵢₛₑ}_{²} and the variance of 1. μ_{σ}_{ₙₒᵢₛₑ}_{²}—a prior estimation of the mean value of the variance of the current noise, varies between 0.1 and 1 depending of the noise in a particular patch clamp setup. p_{i,j}—a probability of transition from i-th conductance level to j-th conductance level during one time step. p_{base}—the base value of pᵢⱼ, if i=0. p_{base} equals 0 if N=1 else 0.05. coef—the coefficient adjusting p_{base} for transitions from higher (starting from 1-nd conductance level) conductance levels, the optimal value of coef for sodium channels analysis is 1. pᵢ—the vector of probabilities of transition from i-th conductance level to each of conductance levels, then A=(pᵢⱼ)—a transition probabilities matrix. I—an observed sequence of current amplitudes over time in an analyzed sweep (Iₜ—a current amplitude at a time point t). HMM ( [symbol] (0, N−1), Cat(pᵢ), [symbol] (μᵢ, σₙₒᵢₛₑ_{ᵢ}²)), —hidden Markov model distribution parametrized with initial distribution, [symbol] (0, N−1), transition distributions Cat(pᵢ) and observation distributions [symbol] (μᵢ, σₙₒᵢₛₑ_{ᵢ}²). [symbol] (0, N−1)—a discrete inform distribution over all conductance levels assuming that at the time point t₀=0 there are equal probabilities for all conductance levels. Cat(pᵢ)—categorical distributions of transitions from i-th conductance level to any of all conductance levels. [symbol] (μᵢ, σₙₒᵢₛₑ_{ᵢ}²)—normal distributions of observing current I(t) given the channel population is at i-th conductance level at a time point t.

For N=1 the model is simplified:

$$\begin{matrix}N = 1 , \\ {μ}_{0} ∼ 𝒩 ( 0 , {σ}_{μ}^{2} ) , \\ {σ}_{n o i s {e}_{0}}^{2} ∼ 𝒩 ( {μ}_{{σ}_{n o ise}^{2}} , 1 ) , \\ p = ( 0 ) , \\ I = ( {I}_{0} , … , {I}_{t} , . . {I}_{T - 1} ) , \\ \\ I ∼ HMM ( 𝒰 ( 0 ) , Cat ( p ) , 𝒩 ( {μ}_{0} , {σ}_{n o {ise}_{0}}^{2} ) ) ,\end{matrix}$$

Next, maximum a posterior estimation is performed with the Adam optimization algorithm (learning rate 0.1, number of optimization steps 60) to infer optimal μ=(μ₀, . . . , μᵢ, . . . , μ_{N-1}) and σₙₒᵢₛₑ²=(σₙₒᵢₛₑ_{₀}², . . . , σₙₒᵢₛₑ_{ᵢ}², . . . , σₙₒᵢₛₑ_{_{N-1}}²) by maximizing the unnormalized posterior density p(μ, σₙₒᵢₛₑ²|I,μᵢₙᵢₜ,σ_{μ}²,μ_{σ}_{ₙₒᵢₛₑ}_{²}):

$$\begin{matrix}p ( μ , {σ}_{n o i s e}^{2} \vert I , {μ}_{i nit} , {σ}_{μ}^{2} , {μ}_{{σ}_{n o i s e}^{2}} ) ∝ p ( I \vert μ , {σ}_{n o ise}^{2} , {μ}_{i nit} , {σ}_{μ}^{2} , {μ}_{{σ}_{n o i s e}^{2}} ) . \\ p ( μ , {σ}_{n o i s e}^{2} \vert {σ}_{μ}^{2} , {μ}_{{σ}_{n o i s e}^{2}} ) , \\ {max}_{μ , {σ}_{noise}^{2}} p ( μ , {σ}_{n o i s e}^{2} \vert I , {μ}_{i nit} , {σ}_{μ}^{2} , {μ}_{{σ}_{n o i s e}^{2}} ) ,\end{matrix}$$

where μᵢₙᵢₜ=(μᵢₙᵢₜ_{₀}, . . . , μᵢₙᵢₜ_{ᵢ}, . . . , μᵢₙᵢₜ_{_{N-1}}).

Maximum a posterior estimation is performed in parallel for several Bayesian models with increasing N=1, 2, . . . Nₘₐₓ. Nₘₐₓ is chosen so that the value is higher by 1 than maximal number of simultaneously open channels observed in at least 100 sweeps from one membrane patch. In practice, Nₘₐₓ=5. Then the optimized Bayesian model with the highest unnormalized posterior probability among all optimized Bayesian models is selected. Next, the Viterbi algorithm is used to restore the most likely sequence of conductance levels given the HMM model HMM ( [symbol] (0,N−1), Cat(pᵢ), [symbol] (μᵢ, σ_{noise,i}²)) of the selected Bayesian model.

Idealization is performed by replacing recorded current I=(I₀, . . . , Iₜ, . . . I_{T-1}), Iₜ—the current at time point t, with the time sequence of inferred mean current amplitudes (λ₀_{₀}, . . . , μᵢ_{ₜ}, . . . , μᵢ_{_{T-1}}), μᵢ_{ₜ} ∈μ, which corresponds to the most likely sequence of conductance levels s=(sᵢ_{₀} . . . , sᵢ_{ₜ}, . . . , sᵢ_{_{T-1}}) according to:

$${s}_{{i}_{t}} = i , \qquad if {μ}_{{i}_{t}} ∈ μ .$$

### Part 6: Detection of Cooperative Gating in Multichannel Recordings

To estimate statistical significance of non-independent (cooperative) gating of M ion channels operating in one membrane patch, first, a number of sweeps containing 0, 1, . . . Mₒ, . . . , M open channels (Mₒ—a number of open channels) for each time point t (L_{M}_{ₒ}(t)) is calculated:

$$\begin{matrix}\begin{matrix}{s}_{w} = ( {s}_{{w}_{0}} , … , {s}_{{w}_{t}} , … , {{s}_{w}}_{T - 1} ) , & {s}_{{w}_{t}} ∈ ( 0 , 1 , … , {s}_{i} , … , N - 1 ) , & i ∈ \{ 0 , 1 , … , N - 1 \} , w ∈\end{matrix} \\ \{ 1 , 2 , … , W \} \\ {m}_{{o}_{w}} ( t ) = {s}_{{w}_{t}} , \\ δ ( {m}_{{o}_{w}} ( t ) ) = \{ \begin{matrix}0 , & if {m}_{{o}_{w}} ( t ) \neq {M}_{o} \\ 1 , & if {m}_{{o}_{w}} ( t ) = {M}_{o}\end{matrix} , \\ {L}_{{M}_{o}} ( t ) = {\sum }_{w = 1}^{W} δ ( {m}_{{o}_{w}} ( t ) ) ,\end{matrix}$$

where w—a current sweep index, W—total number of sweeps, s_{w}—an optimal sequence of conductance levels in w-th sweep inferred at step 5, s_{w}^{t}—conductance level at time point t in a w-th sweep, i—index of a conductance level, N—the total number of conductance levels, mₒ_{_{w}}(t)—a number of open channels in w-th sweep at a time point t, δ(mₒ_{_{w}}(t))—the indicator function equals 0 if mₒ_{_{w}}(t)≠Mₒ or 1 if mₒ_{_{w}}(t)=Mₒ. Thus, at each time point t a tuple (L₀, L₁, . . . , L_{M}_{ₒ}, . . . , L_{M}) is calculated.

Next, gating of a single ion channel m in a population of M ion channels (m∈(1, 2, . . . , M)) is described by a categorical random variable Xₘ defined on a set Ω={O, C} where O—open state, C—closed state. Then joint gating of M ion channels is described by a joint distribution of X1, . . . Xₘ, . . . , X_{M} defined on a set Ω^{M} which is a Cartesian power of Ω={0, C}. Let [symbol] be a tuple within Ω^{M} ( [symbol] ∈Ω^{M}) representing one compound conducting state of a population of M ion channels. For example, for M=4 and ion channels named A, B, C, D, [symbol] can be (OOCC) meaning that channels A and B are in the open state, and channels C and D are in the closed state. Let [symbol] ₘ be an element of [symbol] , then [symbol] ₘ, represents a conducting state (C or O) of a single channel m in a compound conducting state of channels M. If o( [symbol] ) is a number of open channels in a and C(M, o( [symbol] )) is a number of all possible combinations of o( [symbol] ) open channels from M available channels, we can calculate a joint probability p(X₁= [symbol] ₁, . . . , Xₘ= [symbol] ₘ, . . . , X_{M}= [symbol] ₘ) to find each of the channels in a particular conducting state at a time point t assuming that all the channels under a pipette are identical and indistinguishable:

$$\begin{matrix}\begin{matrix}∈ {Ω}^{M} , & {m}_{} ∈ , m ∈ \begin{matrix}( 1 , & … , M ) ,\end{matrix}\end{matrix} \\ C ( M , o ( ) ) = \frac{M !}{( M - o ( ) ) ! o ( ) !} , \\ p ( {X}_{1} = {1}_{} , … , {X}_{m} = {m}_{} , … , {X}_{M} = {m}_{} ) = \frac{1}{{L}_{t o t}} \{ \begin{matrix}\begin{matrix}\frac{{L}_{0}}{C ( M , o ( ) )} , & if o ( ) = 0 , \\ \frac{{L}_{1}}{C ( M , o ( ) )} , & if o ( ) = 1 ,\end{matrix} \\ … , \\ \begin{matrix}\frac{{L}_{{M}_{o}}}{C ( M , o ( ) )} , & if o ( ) = {M}_{o} ,\end{matrix} \\ … , \\ \begin{matrix}\frac{{L}_{M}}{C ( M , o ( ) )} , & if o ( ) = M ,\end{matrix}\end{matrix}\end{matrix}$$

where Lₜₒₜ is the total number of sweeps.

Next, Pearson's χ² test is used to determine statistical significance of independence among X1, . . . Xₘ, . . . , X_{M}. For this purpose, occurrences of all possible combinations of values (joint outcomes) of X1, . . . Xₘ, . . . , X_{M} are expressed in a M-dimentional table having a size of M×M× . . . ×M. Then the table is used to create a contingency table by appending sums of occurrences for rows in each dimension. An occurrence of a particular joint outcome equal a product of a joint probability of this outcome and the total number of all recorded sweeps:

$${L}_{t o t} \cdot p ( {X}_{1} = {1}_{} , … , {X}_{m} = {m}_{} , … , {X}_{M} = {m}_{} )$$

Calculated p value for the null hypothesis (X1, . . . Xₘ, . . . , X_{M} are independent) is used to accept (p>0.05) or reject (p>0.05) the null hypothesis. Since Pearson's χ² test has low power if an occurrence of at least one outcome is below 5. This test is implemented only for that time point where L_{M}(t)=max(L_{M}(t)).

### Part 7: Bayesian Inference of State Transition Probabilities of Discrete Time Markov Models for Single- and Multi-Channel Recordings

The final step of the algorithm is inference of transition probabilities of discrete time Markov models for ion channels. To do this, the total number of ion channels (M) operating under a pipette is calculated:

$$\begin{matrix}\begin{matrix}{s}_{w} = ( {s}_{{w}_{0}} , … , {s}_{{w}_{t}} , … , {{s}_{w}}_{T - 1} ) , & {s}_{{w}_{t}} ∈ ( 0 , 1 , … , {s}_{i} , … , N - 1 ) ,\end{matrix} \\ i ∈ \{ 0 , 1 , … , N - 1 \} , w ∈ ( 1 , 2 , … , W ) , t ∈ ( 0 , 1 , … , t , … , T - 1 ) , \\ {m}_{o} ( w , t ) = {s}_{{w}_{t}} , \\ M = {{max}_{0 \leq t < T ,}}_{1 \leq w \leq W} {m}_{o} ( w , t ) ,\end{matrix}$$

where s_{w} is a vector representation of most likely time sequence of conductance levels in w-th sweep (determined at step 5), N is the total number of conductance levels in all sweeps from one membrane patch, W—the total number of sweeps, t is a time point, T is the total number of time points, mₒ(w, t) is a number of open channels in w-th sweep at a time point t which is equal a conductance level at these sweep and time point. Then M is calculated as the maximal number of open channels observed in in all sweeps over all time points.

Next, for single channel membrane patches the following Bayesian model is used to directly sample transition probabilities given the observed time series of conductance levels in a membrane patch:

$$\begin{matrix}if M = 1 , \\ \begin{matrix}{s}_{w} = ( {s}_{{w}_{0}} , … , {s}_{{w}_{t}} , … , {s}_{{w}_{T - 1}} ) , & {s}_{{w}_{t}} ∈ ( 0 , 1 ) , & i ∈ ( 0 , 1 ) , w ∈ ( 1 , … , W ) ,\end{matrix} \\ π = ( {π}_{0} , … , {π}_{k} , … , {π}_{K - 1} ) , k ∈ ( 0 , … , K - 1 ) , \\ l = ( {l}_{0} , … , {l}_{k} , … , {l}_{K - 1} ) , {l}_{k} ∈ ( 0 , 1 ) , \\ {α}_{k} = ( {α}_{k 0} , … , {α}_{kq} , … , {α}_{k ( K - 1 )} ) , q ∈ ( 0 , … , K - 1 ) , \\ {p}_{k} ∼ Dir ( {α}_{k} ) , \\ {s}_{w} ∼ H M M ( C a t ( π ) , Cat ( {p}_{k} ) , 𝒟 ( l ) ) ,\end{matrix}$$

where k is an index of an ion channel state, K is the total number of states which a single ion channel can be found in. K is the user define parameter, for voltage gated sodium channels typical K varies between 5 and 8. π is a vector of initial probabilities of states, and πₖ is initial probability of k-th state meaning a probability to find a single ion channel in k-th state at time point t=0. Typically, the deepest closed state is set to have πₖ=1, and consequently, all the other states have πₖ=0. l is the vector of conductance levels produced by corresponding single ion channel's state, lₖ is a conductance level produced by a single channel given it is in k-th state. Typically, Markov models of sodium channels have one conducting state. If k-th state is chosen to be conducting state, then lₖ=1, meaning s_{w}_{ₜ}=1 in all sweeps and all time points whenever an ion channels is in k-th state. Consequently, for all other states (named non-conducting states) lₖ=0 meaning that s_{w}_{ₜ}=0 for all sweeps and time points whenever an ion channel is in a non-conducting state. αₖ is a prior concentration of the Dirichlet distribution which is used as a prior distribution of transition probabilities from k-th state to all states (pₖ, pₖ˜Dir(αₖ)). αₖ is a user defined parameter. All αₖ is conveniently organized in a matrix [symbol] of a size K×K:

$$𝒜 = \vert \begin{matrix}{α}_{0} \\ ⋮ \\ {α}_{k} \\ ⋮ \\ {α}_{K - 1}\end{matrix} \vert .$$

s_{w} is modeled as a random vector distributed according to the hidden Markov model distribution HMM(Cat(π), Cat(pₖ), [symbol] (l)) parametrized with the initial categorical distribution [symbol] (0, N−1), the transition categorical distributions Cat(pₖ) and the observation deterministic distribution [symbol] (1l).

For example, to model Naᵥ1.6 channels, the 5 states with 3-rd state is conducting (open) model was chosen. Then the following π, l, and [symbol] were defined:

$$\begin{matrix}π = ( 1 , 0 , 0 , 0 , 0 ) , \\ l = ( 0 , 0 , 1 , 0 , 0 ) , \\ 𝒜 = \vert \begin{matrix}5 , 15 , 0 , 0 , 0 \\ 1 , 1 , 18 , 1 , 0 \\ 0 , 1 , 18 , 0 , 1 \\ 0 , 1 , 0 , 19 , 0 \\ 0 , 0 , 1 , 0 , 19\end{matrix} \vert .\end{matrix}$$

Given A is a K×K matrix of transition probabilities and Σ is a W×T matrix of most likely time sequences of conductance levels in all sweeps:

$$A = \vert \begin{matrix}{p}_{0} \\ ⋮ \\ {p}_{k} \\ ⋮ \\ {p}_{K - 1}\end{matrix} \vert , \qquad Σ = \vert \begin{matrix}{s}_{1} \\ ⋮ \\ {s}_{w} \\ ⋮ \\ {s}_{w}\end{matrix} \vert ,$$

the following unnormalized posterior density:

$$p ( A \vert Σ , 𝒜 ) ∝ p ( Σ \vert A , 𝒜 ) \cdot p ( A \vert 𝒜 ) ,$$

is used to sample values of A with Markov chain Monte Carlo No-U-turn sampler.

The best fit A (denoted as Â) is chosen from all A sampled from the posterior distribution (denoted as Ã) by minimizing the difference between observed and the hidden Markov model predicted ensemble average conductance levels:

$$\begin{matrix}\tilde{A} = \vert \begin{matrix} \\ ⋮ \\ \\ ⋮ \\ \end{matrix} \vert , \\ \vert \tilde{A} ∼ HMM ( Cat ( π ) , Cat ( ) , 𝒟 ( l ) ) , \\ {min}_{\tilde{A}} \{ \vert \frac{1}{\tilde{W}} {\sum }_{\tilde{W} = 1}^{\tilde{W}} \vert \tilde{A} - \frac{1}{W} {\sum }_{w = 1}^{W} {s}_{w} \vert \vert \tilde{A} ∈ \{ {\tilde{A}}_{1} , … , {\tilde{A}}_{H} \} \}\end{matrix}$$

where Ã is a sampled A, [symbol] is a sampled vector of transition probabilities from k-th state into all states, H is the total number of samples, [symbol] Ã is a vector of conductance levels sampled from the hidden Markov model distribution HMM(Cat(π), Cat( [symbol] ) [symbol] (l), \tilde{w}∈(1, 2, . . . , \tilde{W}) is an index of a sample from the hidden Markov model distribution, \tilde{W} is typically set to 10,000.

For membrane patches with more than one active channel s_{w}, π, l, and pₖ are defined as those for single channel models:

$$\begin{matrix}M > 1 , \\ \begin{matrix}{s}_{w} = ( {s}_{{w}_{0}} , … , {s}_{{w}_{t}} , … , {s}_{{w}_{T - 1}} ) , & {s}_{{w}_{t}} ∈ ( 0 , … , {s}_{i} , … , N - 1 ) ,\end{matrix} \\ i ∈ ( 0 , … , N - 1 ) , w ∈ ( 1 , … , W ) , \\ π = ( {π}_{0} , … , {π}_{k} , … , {π}_{K - 1} ) , k ∈ ( 0 , … , K - 1 ) , \\ l = ( {l}_{0} , … , {l}_{k} , … , {l}_{K - 1} ) , {l}_{k} ∈ ( 0 , 1 ) , \\ {α}_{k} = ( {α}_{k 0} , . . , {α}_{kk} , … , {α}_{k ( K - 1 )} ) , \\ {p}_{k} ∼ Dir ( {α}_{k} ) ,\end{matrix}$$

In order to infer behavior of individual ion channels from s_{w}, the channels are assumed to be independent and identical. Then s_{w} is a sum of time sequences of conductance levels of all ion channels operating in a patch. Since one channel can produce only 0 and 1 conductance levels, each s_{w}_{ₜ} value can be presented as a set of 0s and 1s (Ψ_{w}_{ₜ}) such that a number of 1s equals a number of open channels, and a number of 0s equals a number of closed channels. Importantly, since channels are independent and identical the order of 0s and 1s does not matter. Thus, as a first step, s_{w}_{ₜ} is transformed into Ψ_{w}_{ₜ} in the following way:

$$\begin{matrix}{s}_{{w}_{t}} ↦ {Ψ}_{{w}_{t}} ; \\ {Φ}_{{w}_{t}} = \{ φ \vert φ = 0 \} , {Υ}_{{w}_{t}} = \{ υ \vert υ = 1 \} , \\ \begin{matrix}\vert {Φ}_{{w}_{t}} \vert = M - {s}_{{w}_{t}} , & \vert {Υ}_{{w}_{t}} \vert =\end{matrix} {s}_{{w}_{t}} , \\ {Ψ}_{{w}_{t}} = {Φ}_{{w}_{t}} ⋃ {Υ}_{{w}_{t}} .\end{matrix}$$

Next s_{w} is transformed into a matrix ( [symbol] _{w}) of separated time sequences of conductance levels of individual channels m within a w-th sweep (ψ_{w,m}). Thus, ψ_{w,m} is distributed according to the hidden Markov model distribution HMM(Cat(π), Cat(pₖ), [symbol] (l)):

$$\begin{matrix}{s}_{w} ↦ {ℳ}_{w} : \\ \begin{matrix}{ψ}_{w , {m}_{t}} ∈ {Ψ}_{{w}_{t}} , & m ∈ ( 1 , … , M ) ,\end{matrix} \\ {ψ}_{w , m} = ( {ψ}_{w , {m}_{0}} , … , {ψ}_{w , {m}_{t}} , … , {ψ}_{w , {m}_{( T - 1 )}} ) , \\ {ψ}_{w , m} \sim HMM ( Cat ( π ) , Cat ( {p}_{k} ) , 𝒟 ( l ) ) , \\ {ℳ}_{w} = \vert \begin{matrix}{ψ}_{w , 1} \\ ⋮ \\ {ψ}_{w , m} \\ ⋮ \\ {ψ}_{w , M}\end{matrix} \vert .\end{matrix}$$

Next, let z_{w}_{ₜ} be an array equal a cumulative sum of conductance levels of ion channels within one sweep w at one time point t:

$$\begin{matrix}{ψ}_{w , m} ↦ {z}_{{w}_{t}} : \\ {ψ}_{w , {m}_{t}} ∈ {ψ}_{w , m} , \\ {z}_{{w}_{t}} = ( {ψ}_{w , {1}_{t}} , {ψ}_{w , {2}_{t}} + {ψ}_{w , {1}_{t}} , … , {ψ}_{w , {m}_{t}} + {\sum }_{m = 1}^{m - 1} {ψ}_{w , {m}_{t}} , … , {ψ}_{w , {M}_{t}} + {\sum }_{m = 1}^{M - 1} {ψ}_{w , {m}_{t}} ) if t , w = constant .\end{matrix}$$

Then [symbol] is transformed into matrix (Z_{w}) representing time sequences of cumulative sums of individual ion channels in one sweep w:

$$\begin{matrix}{ℳ}_{w} ↦ {Z}_{w} : \\ {Z}_{w} = \vert \begin{matrix}{z}_{{w}_{0}} \\ ⋮ \\ {z}_{{w}_{t}} \\ ⋮ \\ {z}_{{w}_{T - 1}}\end{matrix} \vert .\end{matrix}$$

Thus, Z_{w} is a random matrix distributed according to hidden Markov model distribution ψ_{w,m}˜HMM(Cat(π), Cat(pₖ), [symbol] (l)) transformed by bijective transformation—cumulative sum of ψ_{w,m} over m:

$${Z}_{w} ∼ t H M M ( C a t ( π ) , Cat ( {p}_{k} ) , 𝒟 ( l ) ) .$$

Next, the following unnormalized posterior density:

$$\begin{matrix}Z = \vert \begin{matrix}{Z}_{1} \\ ⋮ \\ {Z}_{w} \\ ⋮ \\ {Z}_{W}\end{matrix} \vert , \\ p ( A \vert Z , 𝒜 ) ∝ p ( Z \vert A , 𝒜 ) \cdot p ( A \vert 𝒜 )\end{matrix}$$

is used to sample values of A with Markov chain Monte Carlo No-U-turn sampler.

Then for each sampled Ã, [symbol] |Ã is calculated as sum of elements of \tilde{Z}_{w}|Ã over m, then the resulting vector is transposed:

$$\begin{matrix}{\tilde{Z}}_{w} \vert \tilde{A} ∼ tHMM ( C a t ( π ) , Cat ( ) , 𝒟 ( l ) ) , \\ \vert \tilde{A} = {( {\sum }_{m = 1}^{M} {\tilde{Z}}_{w} \vert \tilde{A} )}^{T} .\end{matrix}$$

Choosing the best fit A is performed as in single channel recordings:

$${min}_{\tilde{A}} \{ \vert \frac{1}{\tilde{W}} {\sum }_{\tilde{W} = 1}^{\tilde{W}} \vert \tilde{A} - \frac{1}{W} {\sum }_{w = 1}^{W} {s}_{w} \vert \vert \tilde{A} ∈ \{ {\tilde{A}}_{1} , … , {\tilde{A}}_{H} \} \} .$$

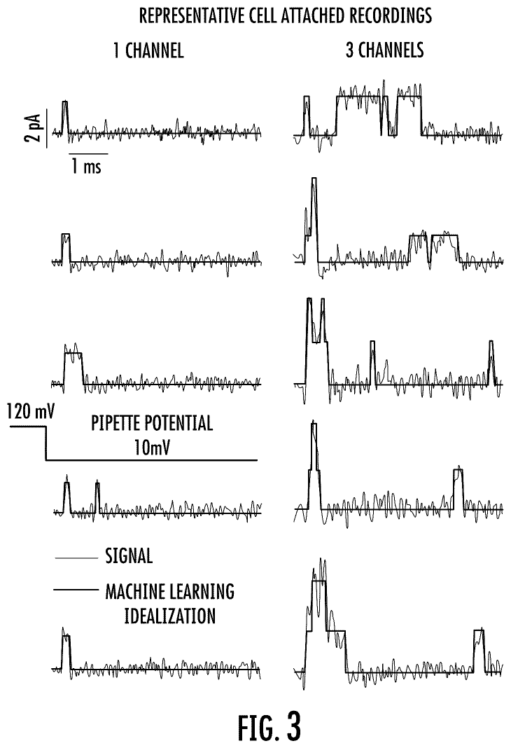

FIG. 3 illustrates representative examples of patch clamp recordings idealization with the Bayesian multiple switch point detection algorithm (machine learning idealization), described in part 5 of the method description. Recordings are obtained from Chinese hamster ovary cell line expressing human (h)Naᵥ1.6 channel according to the part 1 of the method description. Row recordings were then preprocessed (as in part 2), and capacitive currents were subtracted (as described in part 3).

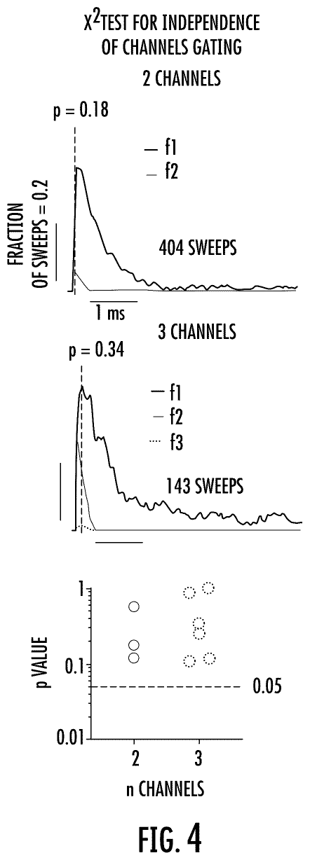

FIG. 4 illustrates detection of cooperative gating in multichannel hNaᵥ1.6 recordings as described in part 6. f1, f2, f3—fractions of current sweeps containing 1, 2 and 3 open channels calculated as L1/Lₜₒₜ, L2/Lₜₒₜ, L3/Lₜₒₜ, respectively, where L1, L2, L3 are numbers of current sweeps with 1, 2 and 3 open channels, and Lₜₒₜ is the total number of recorded sweeps (shown in the figure, upper and middle). Vertical gray dashed lines indicate time points for which X² test of independence of gating was performed. Calculated p values are summarized in the figure (bottom).

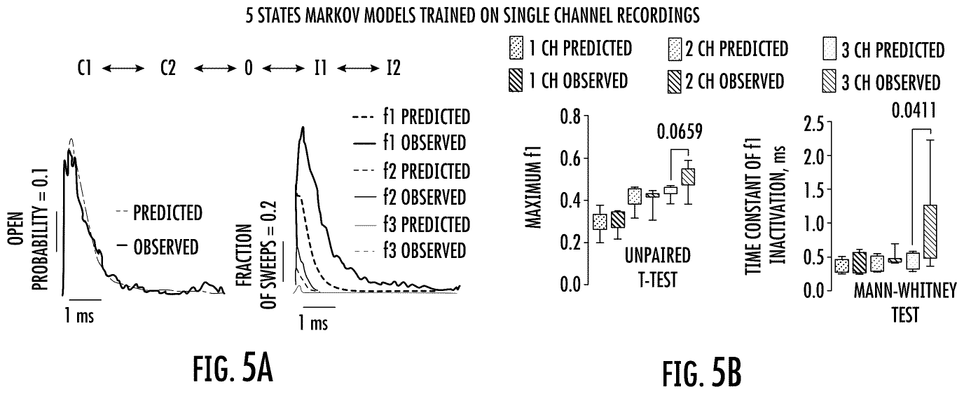

FIGS. 5A-5B illustrate Bayesian inference of state transition probabilities of discrete time Markov models for single hNaᵥ1.6 channel recordings as described in part 7. 5 states model with one open state (shown in the figure) was used. Inferred from single channel recordings transition probabilities matrixes provided a good fit to experimental data of the open probability time course in single channel recordings (FIG. 5A, left). However, prediction of the models differed from experimental data of the f1 time course in 3 channel recordings (FIG. 5A, right). Statistical analysis of maximum of f1 (FIG. 5B, left) and time constant of f1decay (FIG. 5B, right) detected a significant difference comparing single channels model predictions and 3 channel experimental observations. At the same time, there were no differences between observed and predicted f1 for single and two channel recordings. Numbers of analyzed cells: 6, 3, 6 for single, two and three channel recordings, respectively.

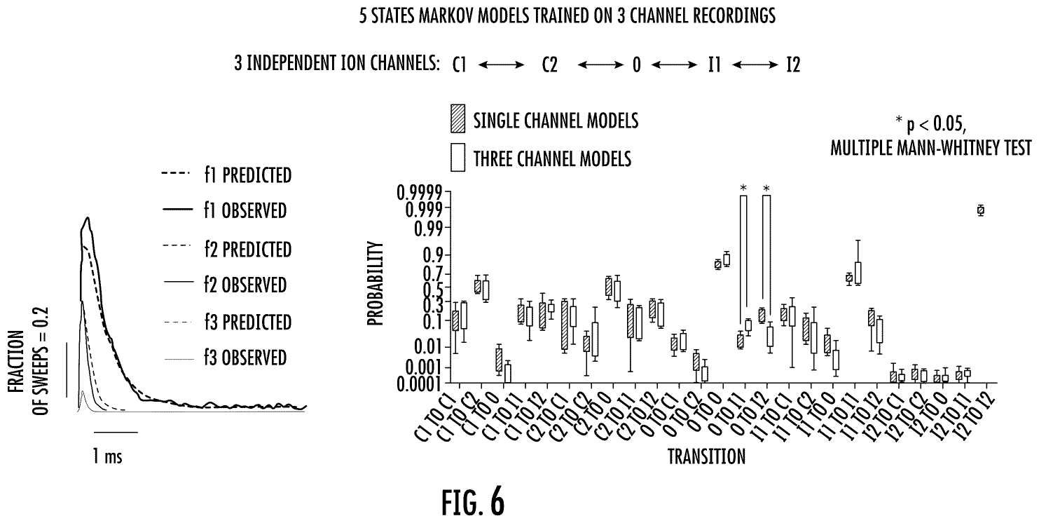

FIG. 6 illustrates Bayesian inference of state transition probabilities of discrete time Markov models for three hNaᵥ1.6 channel recordings as described in part 7. Inferred from three channel recordings transition probabilities matrixes provided a good fit to experimental data of the open probability time course in three channel recordings (left). Comparison of transition probabilities in single and three channel recordings (right) detected significant differences for open (O) to first inactivated (I1) state and open to second inactivated (I2) state transitions. This suggested that inactivation kinetics of h Naᵥ1.6 channels operating in clusters is altered relative to channels operating outside of clusters.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A computer-implemented method comprising:
receiving a multichannel activity signal associated with a plurality of ion channels of a cell;
processing the multichannel activity signal to remove a capacitive transient; and
analyzing the processed multichannel activity signal to assess cooperative ion channel gating behavior for the plurality of ion channels.

2. The computer-implemented method of claim 1, wherein the step of processing the multichannel activity signal to remove the capacitive transient comprises applying a capacitive current subtraction algorithm.

3. The computer-implemented method of claim 1, wherein the step of analyzing the processed multichannel activity signal comprises detecting a time sequence of switches between a plurality of conductance levels.

4. The computer-implemented method of claim 3, wherein the time sequence of switches between the plurality of conductance levels is determined using a Bayesian model.

5. The computer-implemented method of claim 3, wherein the step of analyzing the processed multichannel activity signal further comprises performing a statistical analysis to assess cooperative ion channel gating behavior for the plurality of ion channels, wherein the statistical analysis is based, at least in part, on the time sequence of switches between the plurality of conductance levels.

6. The computer-implemented method of claim 1, further comprising measuring one or more single channel activity amplitudes present in the multichannel activity signal.

7. The computer-implemented method of claim 6, wherein the one or more single channel activity amplitudes are measured by fitting one or more Gaussian mixture models to the multichannel activity signal.

8. The computer-implemented method of claim 1, further comprising inferring a plurality of ion channel gate state transition probabilities of discrete time Markov models for the multichannel activity signal.

9. The computer-implemented method of claim 8, wherein the plurality of ion channel gate state transition probabilities of discrete time Markov models for the multichannel activity signal are inferred using a Bayesian model.

10. The computer-implemented method of claim 1, wherein the multichannel activity signal is measured using a cell-attached patch-clamp system.

11. A method comprising:
applying a drug, compound, or agent to a cell;
recording a multichannel activity signal associated with a plurality of ion channels of the cell, wherein the cell has been exposed to the drug, compound, or agent;
performing the computer-implemented method of claim 1; and
screening the drug, compound, or agent based, at least in part, on the assessed cooperative ion channel gating behavior for the plurality of ion channels.

12. A system comprising:
at least one processor and a memory operably coupled to the at least one processor, the memory having computer-executable instructions stored thereon that, when executed by the at least one processor, cause the at least one processor to:
receive a multichannel activity signal associated with a plurality of ion channels of a cell;
process the multichannel activity signal to remove a capacitive transient; and
analyze the processed multichannel activity signal to assess cooperative ion channel gating behavior for the plurality of ion channels.

13. The system of claim 12, wherein the step of processing the multichannel activity signal to remove the capacitive transient comprises applying a capacitive current subtraction algorithm.

14. The system of claim 12, wherein the step of analyzing the processed multichannel activity signal comprises detecting a time sequence of switches between a plurality of conductance levels.

15. The system of claim 14, wherein the time sequence of switches between the plurality of conductance levels is determined using a Bayesian model.

16. The system of claim 14, wherein the step of analyzing the processed multichannel activity signal further comprises performing a statistical analysis to assess cooperative ion channel gating behavior for the plurality of ion channels, wherein the statistical analysis is based, at least in part, on the time sequence of switches between the plurality of conductance levels.

17. The system of claim 12, wherein the memory has further computer-executable instructions stored thereon that, when executed by the at least one processor, cause the at least one processor to measure one or more single channel activity amplitudes present in the multichannel activity signal.

18. The system of claim 17, wherein the one or more single channel activity amplitudes are measured by fitting one or more Gaussian mixture models to the multichannel activity signal.

19. The system of claim 12, wherein the memory has further computer-executable instructions stored thereon that, when executed by the at least one processor, cause the at least one processor to infer a plurality of ion channel gate state transition probabilities of discrete time Markov models for the multichannel activity signal.

20. The system of claim 19, wherein the plurality of ion channel gate state transition probabilities of discrete time Markov models for the multichannel activity signal are inferred using a Bayesian model.

21. A method comprising:
applying a drug, compound, or agent to a cell;
recording a multichannel activity signal associated with a plurality of ion channels of the cell, wherein the cell has been exposed to the drug, compound, or agent; and
screening the drug, compound, or agent based, at least in part, on a cooperative ion channel gating behavior for the plurality of ion channels.
